(19) United States
(12) Patent Application Publication
(10) Pub. No.: US 2025/0182910 A1
(43) Pub. Date: Jun. 5, 2025

(54) SYSTEMS AND METHODS FOR DISEASE RISK PREDICTION FOR HIGH-RISK PATIENTS USING HIGHEST-K LOSS OPTIMIZED MACHINE LEARNING

(71) Applicant: REGENTS OF THE UNIVERSITY OF MICHIGAN, Ann Arbor, MI (US)

(72) Inventors: Sardar Ansari, Ypsilanti, MI (US); Kevin R. Ward, Superior Township, MI (US); Hongyi Yang, Ann Arbor, MI (US); Alfred Hero, Ann Arbor, MI (US)

(21) Appl. No.: 18/968,898

(22) Filed: Dec. 4, 2024

Related U.S. Application Data
(60) Provisional application No. 63/605,833, filed on Dec. 4, 2023.

(51) Int. Cl. G16H 50/80; G06N 3/04; G16H 50/70

(52) U.S. Cl. CPC G16H 50/80; G06N 3/04; G16H 50/70

(57) ABSTRACT

Systems and methods predict disease risk in a sub-population based on characteristic data including demographic data and health data measured and collected from health monitoring devices. During training, a machine learning model receives biological outcomes for each subject of a population, and is trained using a resource limitation based loss function (“highest-k loss function”) using a soft sorting method to optimize accuracy of the machine learning model for various sub-populations of subjects who are at the highest risk for the biological outcomes. Subsequent data on available resources in a hospital are fed to the trained model for determining biological outcomes for the high-risk sub-population and ranking them for optimizing monitoring, treatment, care, resource allocation.

### CROSS-REFERENCE TO RELATED APPLICATIONS

This application claims priority to U.S. Provisional Application No. 63/605,833, filed Dec. 4, 2023, which is incorporated herein by reference in its entirety.

### STATEMENT OF GOVERNMENT SUPPORT

This invention was made with government support under HL155404 awarded by the National Institutes of Health. The government has certain rights in the invention.

### BACKGROUND

The background description provided herein is for the purpose of generally presenting the context of the disclosure. Work of the presently named inventor, to the extent it is described in this background section, as well as aspects of the description that may not otherwise qualify as prior art at the time of filing, are neither expressly nor impliedly admitted as prior art against the present disclosure.

During the past decades, predicting a patient's disease risk has become an important part of clinical practice. By identifying patients who are likely to have a disease, risk prediction models allow clinicians to design early intervention strategies to prevent disease development. For instance, blood glucose control can decrease microvascular complications risk in prediabetic patients by 25 percent, while early aspirin treatments in patients with myocardial infarction can decrease the risk of mortality from vascular causes by 15 percent, nonfatal myocardial infarction risk by 30 percent, and nonfatal stroke risk by 40 percent.

Despite these advantages, the practical application of disease risk prediction models faces considerable barriers. Due to resource limitations, only a small percentage of patients can be realistically followed up with for disease prevention. For example, 34.5% of U.S. adults have prediabetes, but only 15.3% of them have been informed by a healthcare professional, which is 5.3% of the total population. Although prevailing risk prediction models have achieved good overall performances, they often pay little attention to performance at high-risk levels. It compromises early treatment efficiency and impedes practical uptakes of these risk assessment tools.

There is a need for improved disease risk prediction techniques, in particular techniques that target high-risk patients, as these are the ones most likely to experience the disease and should be prioritized to receive medical treatment in settings with limitations in access to treatment resources.

### SUMMARY OF THE INVENTION

The present disclosure describes systems and techniques that overcome limitations of conventional systems. In various aspects of the present disclosure, systems and techniques are described that perform disease risk prediction that specifically targets high-risk patients. In an aspect, patient disease risk prediction models are developed using a highest-k classification and applying a highest-k loss model that focuses prediction on the subjects having the highest-k predictive scores. The system and techniques deploy a tailored loss function that allows for resource limitation-informed disease risk prediction. This allows for disease risk predictions models that accurately incorporate treatment options and scarcity in those treatment options, thereby improving treatment access and inevitably treatment outcomes.

According to an aspect of the present disclosure, a computer-implemented method for predicting disease risk in a sub-population, the method includes: receiving characteristics data for each subject of a population, the characteristic data comprising demographic data and measured health data for each subject; providing the characteristics data to train a machine learning model, the machine learning model being trained to predict one or more biological outcomes for each subject of the population; during training of the machine learning model, imposing a resource limitation based loss function (“highest-k loss function”) that uses a soft sorting method to optimize accuracy of the machine learning model for a sub-population of the subjects who are at the highest risk for one or more biological outcomes in the population; during utilization of the machine learning model, providing characteristics data on a subsequent population of subjects and subsequent resource limitation data to the machine learning model, and the machine learning model determining one or more biological outcomes for a high-risk sub-population of the subsequent population and ranking the high-risk subpopulation for allocation of resources responsive to the one or more biological outcomes for the high-risk sub-population of the subsequent population; and storing the ranking.

In a variation of this aspect, the machine learning model is a linear regression model.

In a variation of this aspect, the machine learning model is fully connected neural network.

In another variation of this aspect, imposing the resource limitation based loss function during the training of the model includes; performing soft sorting on each of the subjects based on the one or more predicted biological outcomes; iteratively updating a soft sorting parameter; and integrating weights generated from the soft sorting into a loss function.

In a variation of this aspect, the soft sorting algorithm is NeuralSort. In other variations of this aspect, soft sorting algorithm can be Optimal Transport Sort, SoftRank, SoftSort, Fast Soft Sort, Relaxed Bubble Sort, Differentiable Sorting Networks, Monotonic Differentiable Sorting Networks, SmoothI or any other soft sorting method.

### BRIEF DESCRIPTION OF THE DRAWINGS

The figures described below depict various aspects of the system and methods disclosed herein. Each figure depicts an embodiment of a particular aspect of the disclosed system and methods, and each of the figures is intended to accord with a possible embodiment thereof. Further, wherever possible, the following description refers to the reference numerals included in the following figures, in which features depicted in multiple figures are designated with consistent reference numerals.

FIG. 1 is a block diagram of a system architecture for performing high-risk patient disease outcome predictions, in accordance with an example.

FIG. 2 is a block diagram of example high-risk patient prediction computing device as may be used in the system architecture of FIG. 1, in accordance with an example.

FIGS. 3A-3D are plots comparing weights \hat{ω}(s, 25, τ); of the j-th highest score at different j and τ, where s is a random scores vector of size 256 sampled from the uniform distribution over [0, 1], in accordance with an example.

FIGS. 4A-4D are plots of probability density distribution of the positive and negative observations in the two variables of the toy example, in accordance with an example. FIGS. 4A and 4B are the overall distributions. FIGS. 4C and 4D are the zoomed-in distributions on interval [2, 3], in accordance with an example.

FIG. 5 illustrates a nested loop cross-validation process as may be performed by a high-risk patient prediction computing device, in accordance with an example.

FIG. 6 illustrates a nested loop cross-validation architecture that may implement the process of FIG. 5 in a high-risk patient prediction computing device, in accordance with an example

FIG. 7 is a plot of comparing precision at different risk levels between logistic models with the Highest-k loss and the BCE loss on the toy example, in accordance with an example.

FIGS. 8A-8C are plots of inner-testing performances for each combination of model type, loss function, and target proportion K with best hyperparameters from the nested CV, in accordance with an example.

FIGS. 9A-9F are plots of calibration plots of ensemble models with BCE loss, Focal loss, and Highest-k loss on the external set, in accordance with an example.

FIGS. 10A-10D are plots of comparing precision at extremely-high-risk levels among ensemble models with Highest-k loss, BCE loss, and Focal loss on the external set. Differences are the highest kₚ precision of Highest-k loss minus the highest kₚ_precision of BCE or Focal loss. Note that kₚ are represented in permillages, in accordance with an example.

FIG. 11 illustrates a Table I which depicts candidate values for batch size N and target parameter k. k was determined by rounding K×N_so that k observations in the batch correspond to approximately a proportion of all observations that equals K, in accordance with an example.

FIG. 12 illustrates two tables. Table II depicts characteristics of processed datasets, in accordance with an example. Table III depicts trained logistic model weights for the toy example, in accordance with an example.

FIG. 13 illustrates a Table IV which depicts best hyperparameters (τ, k, N) of the highest-k loss from each outer loop of the nested CV with different target proportions K and different model types. Bold fonts indicate that the model was among the seven models with the best performances that were used to compute the ensemble risk scores for the external set, in accordance with an example.

FIG. 14 illustrates two tables. Table V depicts average highest-K PPVs with 95% confidence intervals on outer-testing sets, in accordance with an example. Table VI which depicts highest-K PPVs of ensemble models on the external set, in accordance with an example.

### DETAILED DESCRIPTION

The present disclosure describes systems and methods that overcome limitations of conventional systems. In various aspects of the present disclosure, systems and methods are described that perform disease risk prediction that specifically targets high-risk patients. In an aspect, patient disease risk prediction models are developed using a highest-k classification and applying a highest-k loss model that focuses prediction on the subjects having the highest-k predictive scores. The system and methods deploy a tailored loss function that allows for resource limitation-informed disease risk prediction. This allows for disease risk predictions models that accurately incorporate treatment options and scarcity in those treatment options, thereby improving treatment access and inevitably treatment outcomes.

There are numerous advantages that can be achieved with the present techniques. In hospitals, large numbers of patients must be cared for simultaneously, which requires resource allocations of various staff, physical facilities and space, as well as many different types of monitoring devices, scanning stations, imaging equipment, and other health monitoring stations. Coordination of these resources, especially real time, and with dynamic changes, often determined by different departments and care stations, is technically challenging, and heretofore has not been effectively coordinated with or used in the prediction of high-risk patient outcomes. This is particularly problematic of patients who are most at risk and otherwise most in need of resource allocation. Yet, the present techniques allow for technical solutions to resource allocation, through trained machine learning models tailored to affect better outcomes for high-risk patients using loss optimization during training. This allows for real-time, dynamic ranking of patient sub-populations most at risk and faster or accurate provisioning of healthcare resources to such high-risk sub-populations.

Among these technical advantages, the present techniques provide a centralized (e.g., cloud based) processing system capable of receiving dynamically change resource limitation data, in real-time, and from various different data sources, and using such data in a trained machine learning model-based system for quickly identify and ranking high-risk patients and then allowing for optimized allocation and/or reallocation of those limited resources. In various examples, this optimization of resource allocation includes communicating sub-population rankings and resource allocation instruction data to one or more locations in a hospital for adjusting treatment, monitoring, testing, etc. as provided to a patient.

In various examples, the present techniques deploy systems and methods for predicting disease risk in a high-risk sub-population. These techniques include receiving characteristics data for subject of a population. That characteristic data may be demographic data and measured health data, for example. Other received data includes resource limitation data that defines, for example, the healthcare facilities, healthcare monitoring, and healthcare treatment data that is available or predicted to be available. Both the characteristic data and the resource limitation data are provided to a machine learning model during training of that model. Moreover, during training a resource limitation based loss function is applied to the machine learning model to optimize the trained model for a sub-population of the subjects who are at the highest risk for one or more biological outcomes in the population. In this way, the machine learning model is trained applying a loss function to a high-risk sub-population while excluding subjects of the population not in the high-risk sub-population. The result is a machine learning model trained to rank high-risk subjects, e.g., by performing a highest-K loss minimization, and then assigned subsequent allocation of resources to the high-risk subjects.

While specific examples are discussed, the systems and methods herein may be used to predict any number of targets, such as, for example, disease, condition, diagnosis, prognosis, outcome, clinical trajectory, need for medical treatment, response to treatment, etc. where a provider is interested in focusing analysis on patients with a highest probability score over a general population of patients. In various examples, the probability score used will depend upon the target under analysis. For example, the probability score may be related to the risk of the diseases, the probability of certain outcome, etc. Therefore, while examples relative to risk prediction are described, the present techniques are not limited to risk prediction.

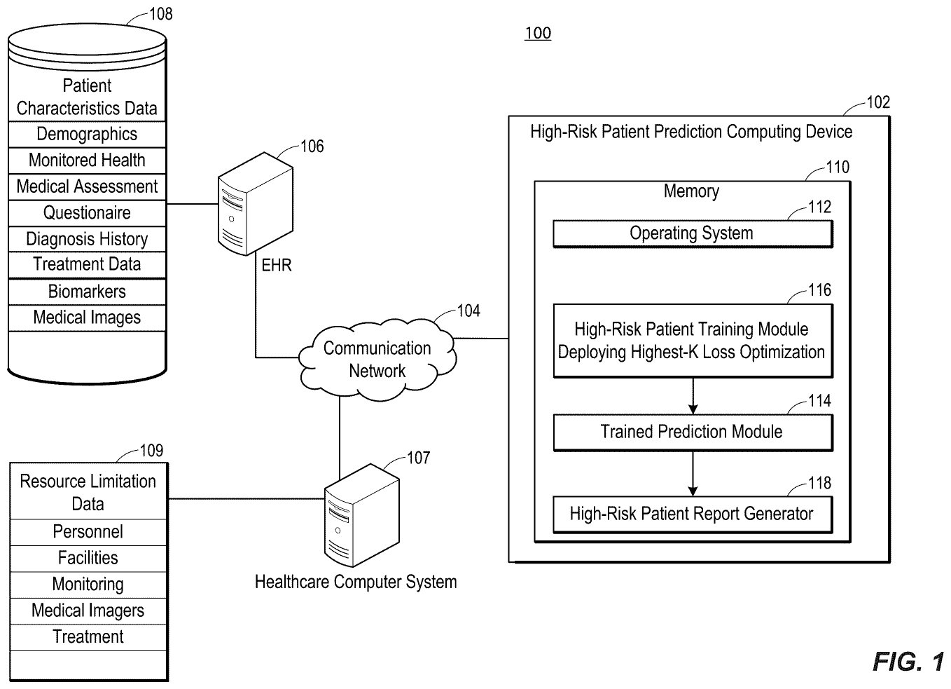

FIG. 1 illustrates a system architecture 100 for performing high-risk patient disease outcome predictions. The system architecture 100 may be used to perform the processes and methods described herein. For example, the system architecture 100 may be used to perform a highest-K loss minimization analysis on one or more predicted biological outcomes of that sub-population and identify a high-risk sub-population of subjects from the overall population of subjects. From the highest-K loss minimization results, the system architecture 100 may generate a report ranking the sub-population, where that ranking may be used by healthcare professions to allocate treatment or other resources based on the predicted biological outcomes. In these ways—and as further described herein—the system architecture 100 applies a loss analysis limited to a most at-risk sub-population, e.g., using a highest-K scores (with k being pre-determined), thereby maximizing prediction model accuracy for the highest risk patients.

In the illustrated example, the system architecture 100 includes a high-risk patient prediction computing device 102 communicatively coupled to one or more different patient data sources through a communication network 104. In the illustrated example, the high-risk patient prediction computing device 102 is coupled to an electronic health records (EHR) system 106 having access to a patient characteristics database 108, storing patient characteristics data 110.

Example characteristics data 110 includes the following example data: demographics data, monitored health data, medical assessment data, medical questionnaire data, diagnosis history, treatment data, biomarker data, and medical image data.

As used herein, “treatment data” may be any type of data corresponding to responsiveness to a subject to medical treatment. For example, if the medical treatment is for a pathology, such as cancer, the treatment data may include cancer treatment efficacy data, including, for example, known drug-gene interaction data, tumor responsive data, known radiation sensitivity biomarkers, immunotherapy biomarkers, combinational therapy biomarkers, drug sensitivity markers or signatures, demographic data, etc.

While the system 106 is described as an EHR system, the system 106 may be any health care provider system e.g., providing access to electronic health records, a computing system access historical medical history data for a large population e.g., including patient outcome data, test results, vital signs, medications, clinical notes, administrative data, treatment team data, location data (medical unit and transfer), medical procedure data, socioeconomic data, environmental factors (e.g., hospital and bed occupancy, nurse workload, number of active orders), etc.

As used herein, “biomarker data” may include (or be derived from) gene expression data in the form of RNA sequencing data (RNASeq), spatial RNASeq, and/or single cell RNASeq (scRNA) data, for example a data store associated with next generation sequencing computer system. Other sources of biomarker data may be transcriptomic data, such as gene pathway data, proteomics data, and/or metabolomics data. More generally, the patient characteristics data 110 may include any number of panomic data for a subject, whereas used herein, the term “panomics” may refer to a range of molecular biology technologies/platforms related to the interaction of biological functions within a cell and with other functions within the human body. For example, panomics may include genomics, epigenomics, chromatin state, transcriptomics, proteomics, metabolomics, biological networks and systems models, etc. Panomic data may be specific to various point in time and to specific tissues and linages of cells, so that panomic data collection is connected to these features and may also be collected and used for a plurality of tissues, lineages, and temporal points connected to phenotypes of interest for a patient. A patient's panomics may relate to biomarkers for multiple phenotypes such as pharmacologic responses to drugs, disease risks, comorbidities, substance abuse problems, etc. Panomics data may be generated and collected for the purpose of a specific set of medical decisions at a discrete point in time and also may be harvested from the sum record of previously collected panomics data at points in the past for an individual patient.

In addition to the EHR system 106, the high-risk patient prediction computing device 102 is connected to a healthcare computing system 107, such as may be used by a hospital system, that has a resources limitation data store 109 storing resource limitation data. For example, the data 109 may store personnel data such as personnel type, expertise, and availability. Other personnel data may include whether the personnel are able to provide telehealth services, in home services, or only at the hospital services. Further the data 109 may store facilities data such as data on available space for admitting a patient and for administering treatment to a patient, location data for determining which patients are in a vicinity of service. Monitoring data, such as available health monitoring equipment and equipment type, may be stored in the data 109, as well. Medical imagers data, such as medical imager type (X-Ray, CT Scan, PET, Ultrasound, etc.) may be stored in the data 109, where such data may include type, availability, and/or usage schedule. Still further data in data 109 may include treatment data, which may be data on type and availability of any of a variety of types of treatments, from drug type and availability, IV availability, etc. The data 109 may be stored in any suitable format and may be coded to identify the data type amongst those shown in FIG. 1 or otherwise.

The high-risk patient prediction computing device 102 may be further connected to various personalized devices (not shown) also connected to the network 104, through which health care professionals and/or patients can enter identification data, request high-risk patient reports from the computing device 102, and initiate other processes described herein. In some examples, these personalized devices may present an instantiation of an accessing (app) to provide such identification data and to allow the devices to be individually authenticated for communication with the computing device 102. These personalized devices may include computing terminal, a laptop computer, a mobile cellular device, and a mobile tablet computing device, by way of example.

In the example of FIG. 1, the system 100 is implemented on a single network accessible server implementing the high-risk patient prediction computing device 102. In other examples, the functions of the high-risk patient prediction computing device 102 may be implemented across distributed devices connected to one another through a communication link. In other examples, functionality of high-risk patient prediction computing device 102 may be distributed across any number of devices, including the portable personal computer, smart phone, electronic document, tablet, desktop personal computer devices shown, and personal mobile and/or monitoring devices, such as wearable devices like smart watches, health rings, and heart rate monitors. In other examples, the functions of the high-risk patient prediction computing device 102 may be cloud-based, such as, for example one or more connected cloud CPU(s) customized to perform machine learning processes and computational techniques herein.

The network 104 may be a public network such as the Internet, private network such as a research institution's private network, a medical healthcare provider's private network, a corporation's private network, or any combination thereof. Networks can include, local area network (LAN), wide area network (WAN), cellular, satellite, or other network infrastructure, whether wireless or wired. The network can utilize communications protocols, including packet-based and/or datagram-based protocols such as internet protocol (IP), transmission control protocol (TCP), user datagram protocol (UDP), Bluetooth, Bluetooth Low Energy, AirPlay, or other types of protocols. Moreover, the network 104 can include a number of devices that facilitate network communications and/or form a hardware basis for the networks, such as switches, routers, gateways, access points (such as a wireless access point as shown), firewalls, base stations, repeaters, backbone devices, etc.

In the illustrated example, the high-risk patient prediction computing device 102 includes a computer-readable memory 110 storing an operating system 112, a trained prediction module 114, a high-risk patient training module utilizing highest-k loss optimization 116, and a high-risk patient report generator 118 to execute various processes described and illustrated herein.

As described in further detailed herein, in various embodiments, the trained prediction module 114 may contain one or more trained machine learning models, such as a linear regression-based machine learning model, a fully connected neural network machine learning model, a convolutional neural network machine learning model, a recurrent neural network machine learning model, a transformer machine learning model, an attention-based machine learning model, a decision tree machine learning model, a random forest machine learning model, a gradient boosting trees machine learning model, trained to predict a disease state of a subject, or any other type of machine learning module that is trained using a loss function and generates a prediction score, to predict a disease, condition, diagnosis, prognosis, outcome, clinical trajectory, need for medical treatment or response to treatment, or any other prediction target, for a patient. In various examples, the trained prediction module 114 may include any machine learning models trained using one or more types of patient characteristic data, such as the data 110, and generates classification data, such as one or more disease predictions, for a population of subjects.

Further, however, in accordance with the techniques herein, these machine learning models of the trained prediction module 114 are trained through the high-risk patient training module 116.

In an example, during machine learning model training, the training module 116 receives resource limitation data 109 and uses a highest-k loss optimization to train machine learning models, to identify a subpopulation of high-risk patients and rank the subpopulation based on a risk profile. In various examples, the operation of the high-risk patient training module 116 occurs during training resulting in the trained prediction module 114 having one or more trained models generating a predicted outcomes of a subpopulation of high risk patients, where those predicted outcomes may be reported out by the high-risk patient report generator 118. While the high-risk patient training module 116 is shown within the computing device 102, in other examples the training module 116 may be deployed in a separating computing device from that storing the resulting trained prediction models.

Thus, in some examples, machine learning model training includes receiving characteristics data for subjects of a population and imposing, during training, a resource limitation based loss function (also called a “highest-k loss function” herein) that uses a soft sorting method to optimize accuracy of the machine learning model for a sub-population who are at the highest risk for one or more biological outcomes. In some examples, imposing the resource limitation based loss function during the training of the model includes (i) performing soft sorting on each of the subjects based on the risk or probability of one or more predicted biological outcomes, (ii) iteratively updating a soft sorting parameter to approach a sorted list of patients based on the risk or probability of one or more predicted biological outcomes, and (iii) integrating weights generated from the soft sorting into a loss function. Thus, machine learning training includes, in some examples, gradually going from a soft sorting to a hard sorting where loss optimization based on both characteristics data and resource limitation based data is used for training model predictions.

During the application and utilization stages, the trained prediction module 114, receives patient characteristics data from the database 108 for each subject of a population. That population may include only current patients of one or more healthcare providers, that is, patients for whom the provider desires to monitor and track for predicting disease, predicting treatment needs, or predicting other potential interactions with the healthcare provider, such as, routine medical checkups or screening. The high-risk patient prediction method may further include providing that patient characteristic data to the one or more trained prediction module 114. The trained prediction module 114 may then generate one or more predicted biological outcomes for each subject of the population. For example, biological outcomes are predicted for each subject of a high-risk sub-population of a larger population group, in instances where data for a larger population group is provided to the trained prediction module 114.

Further, to ensure that the predicted outcome values of the high risk patients are determined against limitations in resources of healthcare providers/facilities, during application and utilization stages, the trained prediction module 114 is pre-trained to impose a resource limitation model on the one or more biological outcomes generated to identify a sub-population of the subjects in the population, these subjects correspond to high-risk subjects, where their risk is assessed based on both the predicted biological outcomes and the resources limitations of the healthcare computing system 107. Further, the trained prediction module 114 deploys a trained machine learning model that is trained using highest-k loss optimization to identify subjects in the high-risk sub-population and exclude subjects of the population not in the high-risk sub-population for one or more biological outcomes of the sub-population. After applying a trained machine learning model that is trained using the highest-K loss minimization, the high-risk subjects are ranked in order of disease risk, and the ranking is stored by and a report generated by the high-risk patient report generator 118 for allowing the healthcare computing system 107 to allocate resources for receiving the high-risk population, to allocate resources initiating interaction with the high-risk population, to communicate messages to the high-risk population, to communication messages to healthcare providers for the high-risk population, and the like.

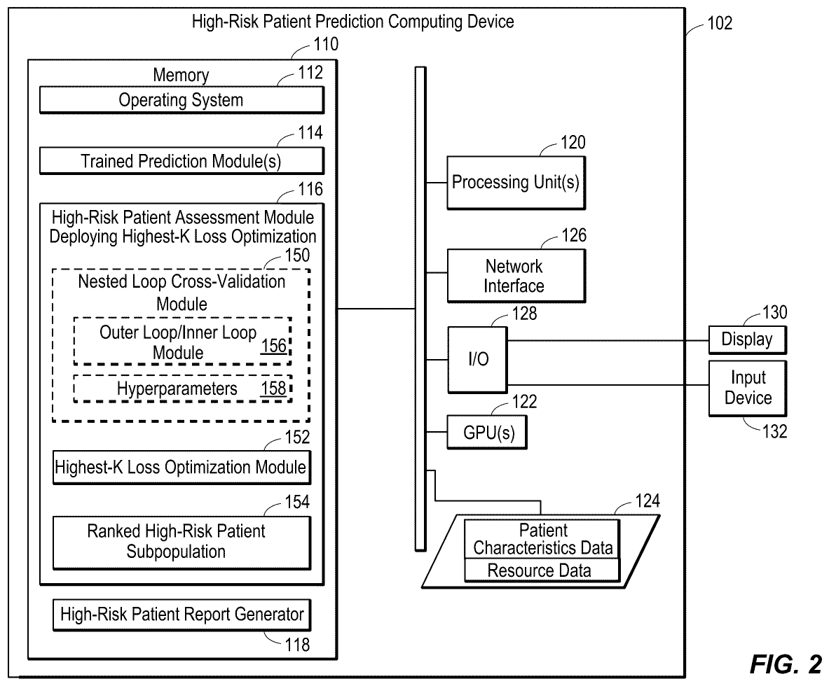

FIG. 2 illustrates a detailed example implementation of the high-risk patient prediction computing device 102 of FIG. 1. As such, the computing device 102 includes a communication bus 119 communicatively coupled with one or more processing units 120, one or more optional graphics processing units 122, a local database 124, the computer-readable memory 110, a network interface 126, and Input/Output (I/O) interfaces 128 connecting the computing device 102 to a display 130 and user input device 132.

The computer-readable media 110 may include executable computer-readable code stored thereon for programming a computer (e.g., comprising a processor(s) and GPU(s)) to the techniques herein. Examples of such computer-readable storage media include a hard disk, a solid state storage device/media, a CD-ROM, digital versatile disks (DVDs), an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory) and a Flash memory. More generally, the processing units of the computing device 102 may represent a CPU-type processing unit, a GPU-type processing unit, a field-programmable gate array (FPGA), another class of digital signal processor (DSP), or other hardware logic components that can be driven by a CPU.

In various examples, the computing device 102 implements highest-k loss minimization processes to machine learning models to identify a subpopulation at high risk of disease and rank that subpopulation based on disease prediction in a resource constrained healthcare system. Using a highest-k loss minimization process during training allows the resulting trained models to focus on patient subpopulations with highest risk and therefore achieve higher prediction accuracy in those subpopulations, in contrary to conventional machine learning methods that focus on the entire patient population irrespective of the patient's risk of developing the disease/condition. For example, rapid response teams (RRT) use risk prediction models to identify patients that are at high risk of clinical deterioration and proactively round on those patients to treat them and prevent their deterioration. However due to resource limitations (e.g., the number of RRT nurses available), the proactive rounding can only be carried out for a limited number of patients a day, say k. As a result, the accuracy of the risk prediction model is only relevant for patients who receive the highest k scores. In other words, how accurate the model is in predicting the risk for the remaining patients (i.e., those whose score is not among the patients with highest k scores) is inconsequential since they are unlikely to receive treatment (RRT rounding) due to resource limitations. In another example, hospital readmission risk models are used to identify patients at a high risk of readmission to prevent the readmission. For example, the healthcare system may allocate a certain number of nurses to contact patients at a high risk of readmission to follow up on their condition and ensure they are taking their medications and following clinical recommendations. However, there is often a limit on the number of nurses that are available for follow up; hence, not every patient can be followed up on. Given the maximum number of patients that can be followed up on in a certain period of time (e.g., k patients a day) given the existing resource limitations, the highest-k loss risk prediction model can be trained to maximize the model accuracy for k patients who receive the highest scores. This may result in a lower accuracy for the remaining patients (those who are not among the subpopulation with the highest-k scores), but this is inconsequential because those patients are unlikely to receive a follow up given resource limitation. This is in contrast to existing classification methods that optimize the prediction accuracy for all patients during model training, while the highest-k loss allows the training to focus on patients for whom the prediction accuracy is consequential. It is worth noting that while existing classification methods allow for patients to be assigned different weights during model training, these weights cannot be used to train a model that is more accurate on the patients with highest scores because the prediction scores are not know a priori at training time.

The Highest-k Loss techniques developed herein, and applied by the training module 116, are designed to address the risk prediction scenario in which healthcare providers wish to focus on the highest k predictive scores. One potential solution would be to sort the predictive scores and apply a loss function only to the highest k scores. Let P(s)∈{0,1}^{n×n} be a permutation sorting matrix that sorts the vector s∈ [symbol] ⁿ and is defined as:

$${P ( s )}_{ij} = \{ \begin{matrix}1 , & if {s}_{j} is i - th highest score ; \\ 0 , & otherwise .\end{matrix} \qquad ( 1 )$$

Then, P(s)s will be the sorted version of s. From that, we can obtain a weights vector w(s, k)∈ [symbol] ⁿ indicating which scores are among the highest k by summing up the first k rows of the sorting matrix:

$${w ( s , k )}_{j} = {\sum }_{i = 1}^{k} {P ( s )}_{ij} = \{ \begin{matrix}1 , if {s}_{j} is among highest k scores ; \\ 0 , otherwise .\end{matrix} \qquad ( 2 )$$

However, the sorting operation is non-differentiable, which prohibits gradient-based optimization. Therefore, for exemplary solutions herein instead of using a non-differentiable sorting operation, we have developed a differentiable estimation. For example, we initially look to NeuralSort, which is a continuous relaxation of the sorting operator to the set of unimodal row-stochastic matrices. Let \hat{P}(s, τ) denote the NeuralSort estimation of the sorting matrix, where τ>0 is a temperature parameter. The i-th row of \hat{P}(s, τ), denoted by \hat{P}(s, τ)ᵢ, is given by:

$${\hat{P} ( s , τ )}_{i} . = softmax ( \frac{( n + 1 - 2 i ) s - As 𝕝}{τ} ) , \qquad ( 3 )$$

where Aₛ is the absolute pairwise differences matrix of scores defined by A_{s,ij}:=|sᵢ−sⱼ|. As the temperature parameter approaches zero, the NeuralSort estimation converges to the sorting matrix:

$${lim}_{τ \to {0}^{+}} \hat{P} ( s , τ ) = P ( s ) . \qquad ( 4 )$$

The NeuralSort algorithm is defined row-wise, so we can also obtain a weights vector ŵ(s, κ, τ) by summing up the first k rows of the NeuralSort estimation:

$${\hat{w} ( s , k , τ )}_{j} = {\sum }_{i = 1}^{k} {\hat{P} ( s , τ )}_{ij} . \qquad ( 5 )$$

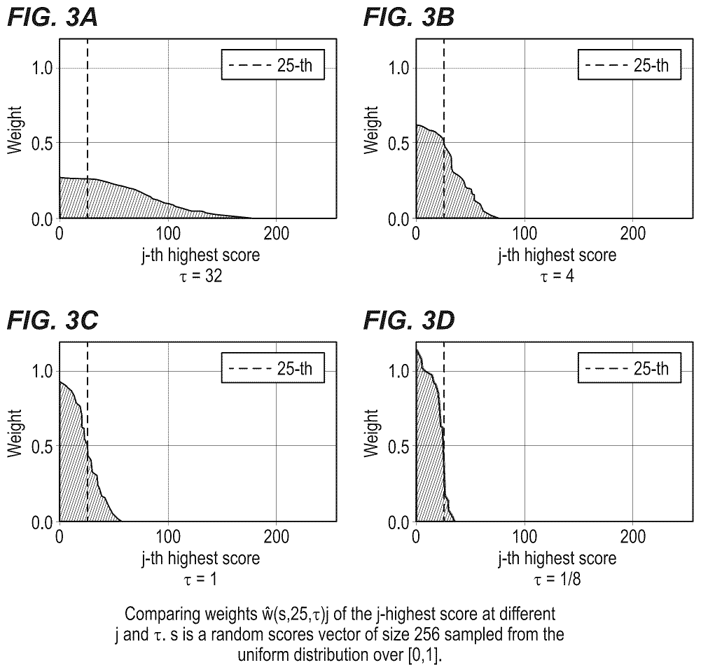

FIGS. 3A-3D shows the weights in ŵ at different values of τ using randomly generated scores. As τ approaches zero, weights for the highest k scores approach 1 and the rest of the weights approach 0. It is worth noting that the NeuralSort approach was used in the described example, but that NeuralSort can be replaced by any other soft sorting algorithm approach in the Highest-k loss to achieve the same effect, i.e., forcing the optimization to focus on the k patients with the highest k score values. NeuralSort is an example of a soft sorting approach. Any suitable soft sorting approach may be used herein allowing for probabilistic sorting of data.

Since we only care about minimizing the number of negative observations in the highest k scores (i.e., false positives among the subjects with the highest k scores), we define its weighted average as the Highest-k loss:

$${ℒ}_{k , τ} ( s , y ) = \frac{{\sum }_{j = 1}^{N} {\hat{w} ( s , k , τ )}_{j} ( 1 - {y}_{j} )}{{\sum }_{j = 1}^{N} {\hat{w} ( s , k , τ )}_{j}} , \qquad ( 6 )$$

where k∈{1, 2, . . . , N} is called the target parameter, τ is the temperature parameter, N is the batch size, s∈[0, 1]^{N} is the predicted risk scores vector outputted by the risk prediction model, and y∈{0, 1}^{N} is the ground-truth labels vector. The target parameter k represents the number of observations in each training batch that the Highest-k loss focuses on. Alternatively, the weights ŵ(s, k, τ) can be used as observation weights in any conventional classification loss function such as the binary cross-entropy (BCE) loss or the Focal loss (both defined below), i.e., the terms associated with each observation in the loss function can be weighted by ŵ(s, k, τ) to force the optimization towards minimizing the loss for the k patients with highest scores and eliminating the effect of the other observations on the optimization. As a result, any existing classification loss can be adjusted to become a highest-k loss by incorporating the weights ŵ(s, k, τ) into the loss function. For example, in the case of BCE, the highest-k loss can be written as.

$${ℒ}_{BCE , k , τ} ( s , y ) = - \frac{{\sum }_{j = 1}^{N} {\hat{w} ( s , k , τ )}_{j} [ {y}_{j} log ( {s}_{j} ) + ( 1 - {y}_{j} ) log ( 1 - {s}_{j} ) ]}{{\sum }_{j = 1}^{N} {\hat{w} ( s , k , τ )}_{j}}$$

The above expression is another example of converting an existing classification loss function into a highest-k loss function, satisfying the techniques herein.

To provide a comparison of the highest-k loss techniques herein and other loss functions, we developed the following evaluation processes. Our baseline loss functions are BCE loss and Focal loss. Cross-entropy loss is the most commonly used loss function in classification tasks:

$${ℒ}_{BCE} ( s , y ) = - \frac{1}{N} {\sum }_{j = 1}^{N} [ {y}_{j} log ( {s}_{j} ) + ( 1 - {y}_{j} ) log ( 1 - {s}_{j} ) ] . \qquad ( 7 )$$

Focal loss is a variant of the cross-entropy loss that considers the class imbalance and focuses the learning on the difficult minority samples:

$${ℒ}_{γ , α} ( s , y ) = - \frac{1}{N} {\sum }_{j = 1}^{N} [ {α ( 1 - {s}_{j} )}^{γ} {y}_{j} log ( {s}_{j} ) + ( 1 - α ) {s}_{j}^{γ} ( 1 - {y}_{j} ) log ( 1 - {s}_{j} ) ] , \qquad ( 8 )$$

where γ is the focusing parameter that reduces the loss contribution from easy samples, and α is the balancing factor that weights positive and negative samples.

All loss functions were applied to the logistic model and the fully connected neural networks (FCNN). The logistic regression model can be represented by:

$$s = σ ( {β}_{0} + {\sum }_{i = 1}^{m} {β}_{i} {x}_{i} ) , \qquad ( 9 )$$

where s is the predictive score (model output). βᵢ are the model parameters to be learned from training data, xᵢ∈ [symbol] ^{m} is the i-th input observation with m feature variables, and σ is the sigmoid function. The FCNN model has one hidden layer with 10 units and can be represented by:

$$s = σ ( {β}_{0} + {\sum }_{i = 1}^{10} {β}_{i} {z}_{i} ) , \qquad ( 10 )$$

$${z}_{i} = Re LU ( {β}_{i 0} + {\sum }_{j = 1}^{m} {β}_{ij} {x}_{j} ) , \qquad ( 11 )$$

where z is the hidden layer vector.

The performances of models were evaluated by the Highest-kₚ, PPV, defined as the positive predictive value (i.e., precision) on the highest kₚ predictive risk scores.

### Example Implementation I—Toy Example

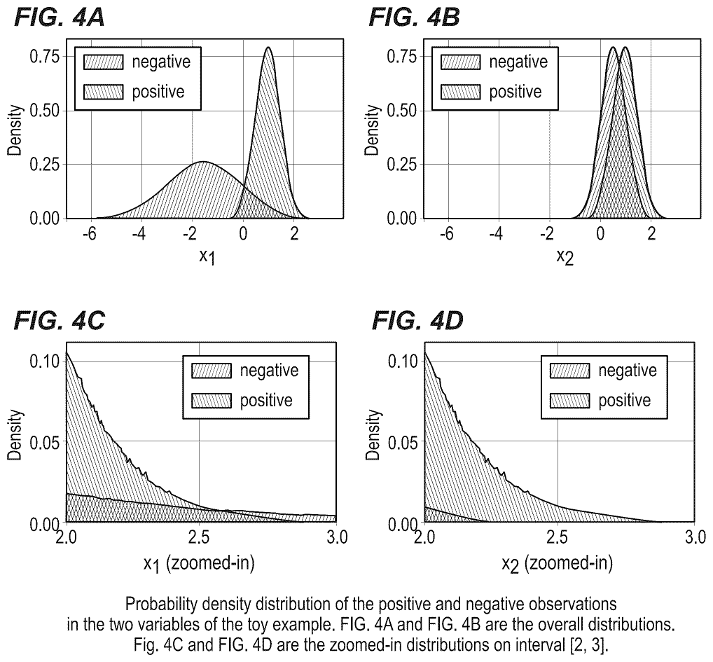

In configuring the high-risk patient module deploying highest-k loss optimization 116, we performed testing experiments starting with a simple toy example with two predictors, x₁ and x₂, both generated by a normal distribution as shown in FIGS. 4A-4D. A total of 100,000 observations were used to train models and 10,000 observations were used to evaluate the model performances. For both the training and testing sets, the event rate was set to 50%. Since the stimulated data are balanced and have only two predictors, we just evaluated the logistic model with the Highest-k loss and the BCE loss. The distributions of the positive and negative classes are less overlapped in the x₁ variable, therefore x₁ has a higher discrimination power and is a more important factor in the classification. However, if we focus on the precision at high-risk levels, x₂ would be a better predictor because almost all samples are positive when x₂>2.3.

In the illustrated example of FIG. 2, the high-risk patient training module 116 (deploying highest-k loss optimization) was validated using an optional nested loop cross-validation module 150, a highest-k loss optimization module 152, and resulting ranked high-risk patient subpopulation data 154. To implement nested loop cross-validations, the module 150 includes an outer loop/inner loop module 156 and generated hyperparameters 158. Example operations of the nested loop cross-validation module 150 and the highest-k loss optimization module 152, which collectively performed a nested cross-validation and model aggregation, are now described.

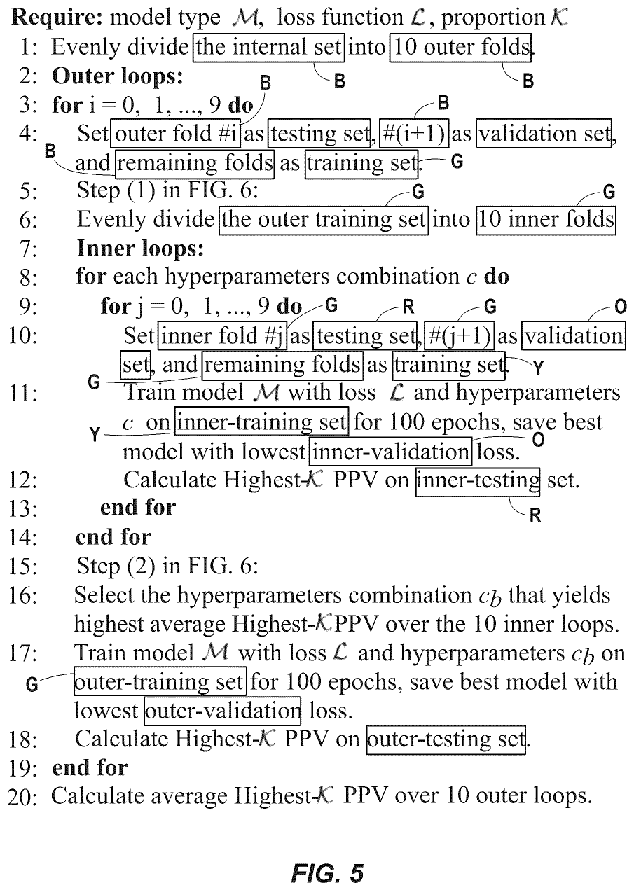

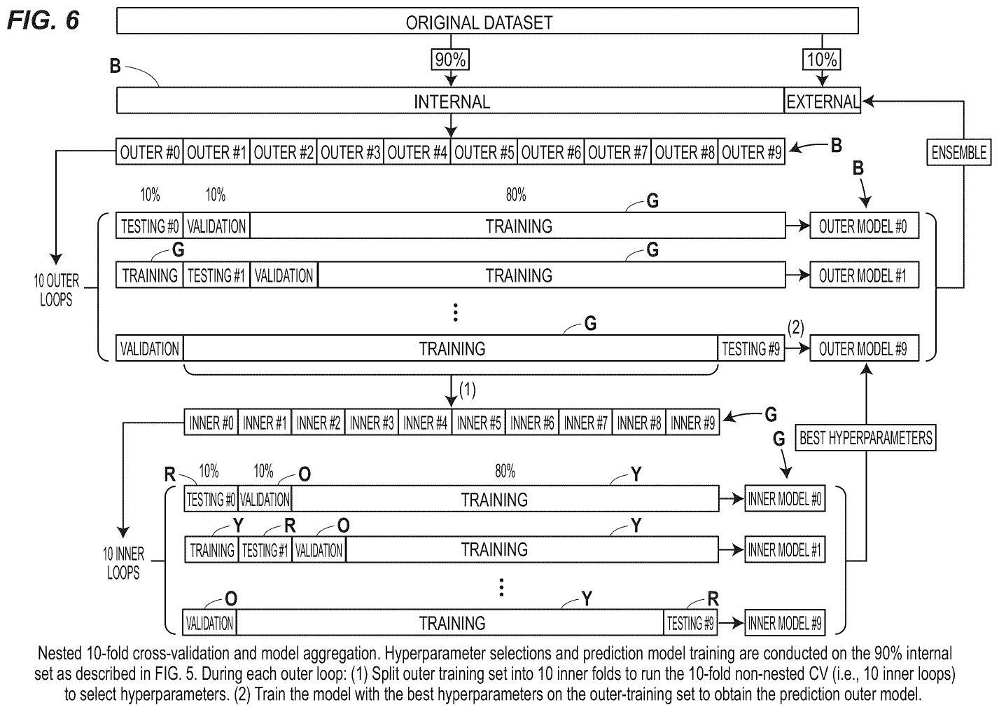

To evaluate the Highest-k loss, in an example configuration of the module 152, we ran nested 10-fold cross-validation (CV) on an internal set (90% of the full dataset). In this example, the configuration includes 10 outer loops with 10 inner loops each. At each outer loop, 10 inner loops performed cross-validation to identify the best hyperparameters. 10 outer loops were executed each time a nested CV was run, resulting in 10 prediction outer models. 18 combinations of 2 model types (M∈{logistic, FCNN}), 3 loss functions (L∈{BCE, Focal, Highest-k}), and 3 target proportions (K∈{1%, 5%, 10%}) were tried as settings of the nested CV. The target proportion K represents the proportion of observations we focus on and is determined based on the clinical context, e.g., the maximum number of patients that a healthcare system can follow up with to prevent the target disease in a given period of time. FIG. 5 illustrates an example implementation of a nested loop cross-validation process 200. FIG. 6 illustrates an example nested loop cross-validation architecture 300 for implementing the example process 200 or other processes of the nested loop cross-validation module 150. The portions of the architecture 300 that perform various options in the process 200 are illustrated with letters, B, G, Y, R, and O. In the illustrated example, the architecture 300 illustrates a nested 10-fold cross-validation and model aggregation. Hyperparameter selections and prediction model training are conducted on the 90% internal set as described in the process 200 of FIG. 5. During each outer loop (1) Split outer training set into 10 inner folds to run the 10-fold non-nested CV (i.e., 10 inner loops) to select hyperparameters. And (2) Train the model with the best hyperparameters on the outer-training set to obtain the prediction outer model. FIGS. 5 and 6 provide examples of validating the models herein. Any number of other appropriate validations may be used.

The prediction outer models were further aggregated and evaluated on the external set (10% of the full dataset kept untouched during the nested CV). For each combination of nested CV settings (M, L, K), we picked seven prediction outer models with the best performance (Highest-K PPV) on the outer test sets. Each picked model was then calibrated using Isotonic regression applied to the outer-testing set. Finally, we calculated the median score from the seven calibrated models to compute the ensemble risk score for each sample in the external set.

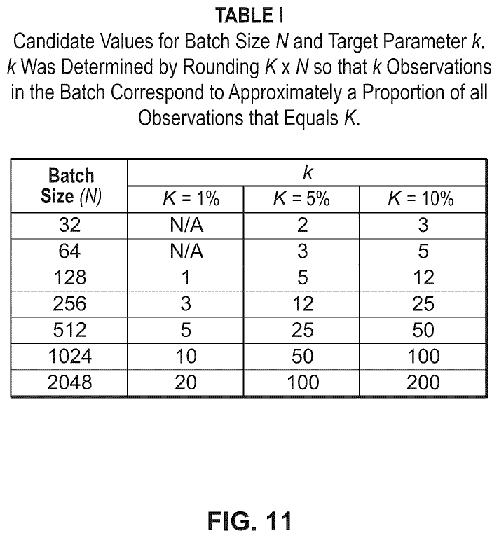

In various examples, the nested loop cross-validation determines inner loop hyperparameters each inner loop execution and outer loop hyperparameters each outer loop execution, where all the hyperparameter sets may be stored as the hyperparameters 158. In an example, the hyperparameters include training batch size N and loss function parameters. Candidate values of the batch size N are shown in the first column of Table I (see, FIG. 11). The BCE loss does not have loss function parameters. For the Focal loss, the focusing parameter γ and the balancing factor α were fixed to 2 and 0.75 respectively. Loss function parameters of the Highest-k loss were the temperature parameter τ and the target parameter k. We chose the initial value of τ from {2, 4, 8, 16, 32} and halved t every 20 training epochs. The value of k was determined by k≈K×N to optimize training losses at the target proportion of observations that we focus on, as shown in Table I (see, FIG. 11). For example, for N=128 and K=1%, k was set to 1. It is worth noting that the approach described above for decreasing τ to gradually approach from soft sorting to hard sorting is just an example and can be replaced by any other appropriate approach.

To compare the Highest-k loss and the baseline loss (BCE or Focal), we also conducted statistical hypothesis tests on the outer-testing results for each K:

$${H}_{0} : {μ}_{d} \leq 0 v . s . {H}_{a} : {μ}_{d} > 0 \qquad ( 12 )$$

where μ_{d} is the mean difference in Highest-K PPV between Highest-k loss and the baseline loss. The null hypothesis H₀ represents that the Highest-k loss does not perform better than the baseline loss, while the alternative hypothesis H_{d} represents that the Highest-k loss performs better. We performed a right-tailed paired t-test to analyze the statistical significance.

To ensure the practical applicability of the Highest-k loss, we also evaluated the Highest-k loss at extremely-high-risk levels. We obtained the ensemble risk scores for each sample in the external set via nested 10-fold CV and model aggregation with K=0.1%. Hyperparameters were similar as previously described with the only difference being that the target parameter k of the Highest-k loss did not need to match the target proportion K. For example, for batch size N=128, any of 1, 5, and 12 could be chosen as the value of k in this section. The reason is that k≈K×N is not always usable, especially when we are focusing on such a small K, e.g., for N=128 and K=0.1%, K×N=0.128≈0.

### Example Implementation II—Diabetes Risk-Factor Data

In an example experiment, we processed Behavioral Risk Factor Surveillance System (BRFSS) survey data to extract binary prediction tasks for examining operation of the high-risk patient module deploying highest-k loss optimization 116. BRFSS is a health-related telephone survey collected annually by the Centers for Disease Control and Prevention (CDC). It collects over 400,000 U.S. residents' health-related risk behaviors, chronic health conditions, and use of preventive services each year. Survey data applied in our study contains 441,456 responses from distinct respondents with 330 features collected in 2015.

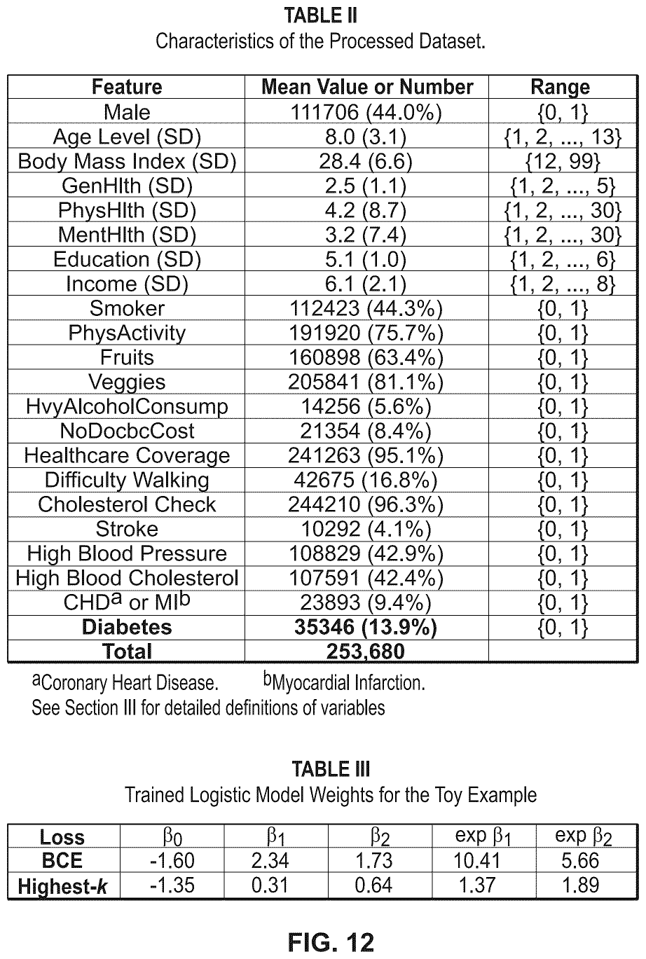

Based on previous research, 21 important risk factors for diabetes were selected as predictors. Survey responses were excluded if any of the selected predictors was missing. The processed dataset contains 253,680 survey responses, 35,346 (13.93%) of which with diabetes or prediabetes were defined as positive samples. Table II (see, FIG. 12) shows the patient characteristic data of the processed dataset. Age is the age level where 1 is the youngest (18 to 24 years old) while 13 is the oldest (80 years old or older). GenHlth is the general health level where 1 is the best while 5 is the worst. PhysHlth/MentHlth is the number of days in the past 30 days that physical/mental health was not good. Education is the education level where 1 is the lowest (never attend school) while 6 is the highest (college graduate). Income is the annual household income level where 1 is the lowest (less than 10,000 dollars) while 8 is the highest (75,000 dollars or more). Detailed definitions of each level in these categorical variables can be found in the BRFSS Codebook Report. Smoker is defined as having smoked at least 100 cigarettes in the entire life. PhysActivity is defined as having done physical activity or exercise during the past 30 days other than the regular job. Fruits/Veggies is defined as consuming fruits/vegetables more than once per day. HvyAlcoholConsum is defined as having more than 14 (male) or 7 (female) drinks per week. NoDocbcCost is defined as having a time in the past 12 months that could not see a doctor because of cost.

### Performance Results for Example Implementations I and II

Toy Table. Table III (see, FIG. 12) shows values of logistic model weights trained on the toy example data using a traditional BCE loss technique and using an example Highest-k loss technique herein. As expected, traditional BCE loss assigned more weight to x1 to maximize the performance across the entire dataset, while our Highest-k loss assigned more weight to x2 to prioritize the performance at high-risk levels.

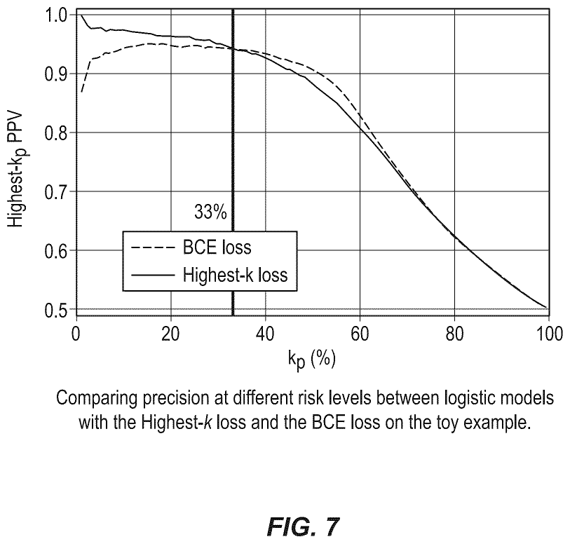

As FIG. 7 shows, the Highest-k loss performed better than the BCE loss for observations with the highest kₚ scores when kₚ≤33%. For very few numbers of high-risk samples when kₚ approaches zero, the Highest-k loss almost achieved 100% precision. To the contrary, the model with the Highest-k loss achieved a lower PPV for observations with lower scores, corresponding to higher values of kp. This can be thought of as the cost associated with improving the PPV for the observations with higher scores. However, as previously discussed, our approach assumes that the inferior performance on observations with the lower scores is inconsequential since providers will never follow up with these patients due to resource limitations.

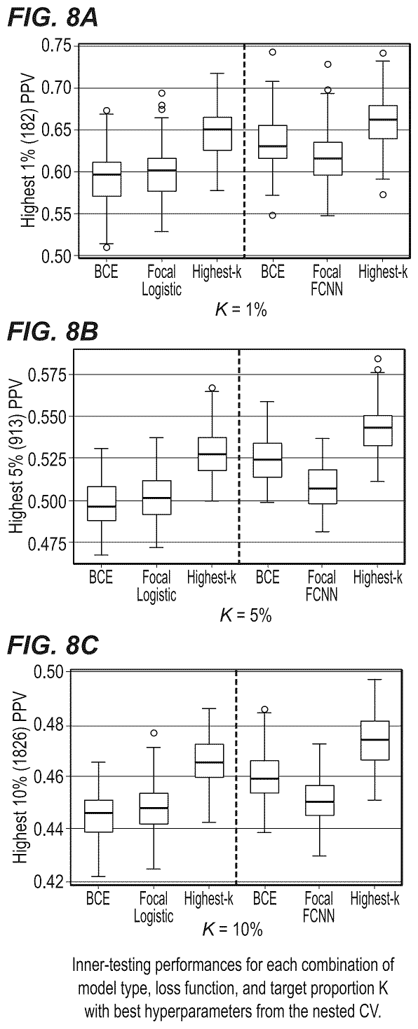

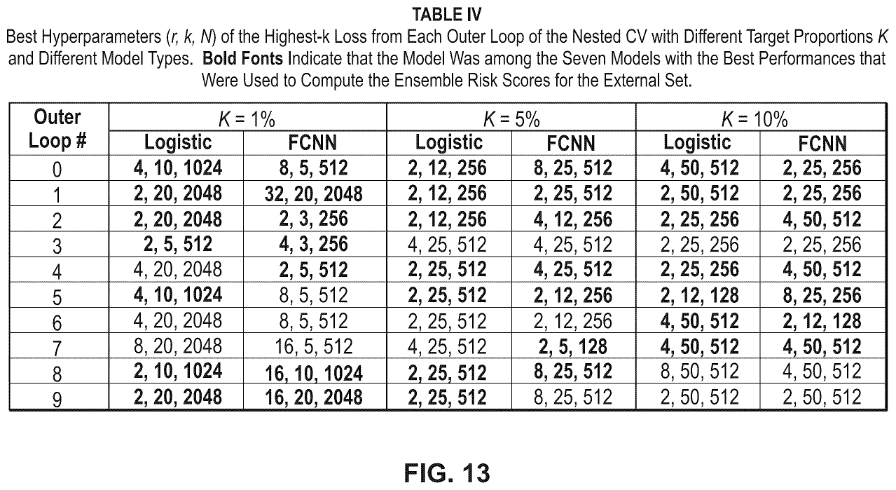

To validate that the Highest-k loss works at its target high-risk level, we evaluated its performances with different k values and the matched target proportion K. Table IV (see, FIG. 13) shows the best hyperparameters of the Highest-k loss from each outer loop in the nested CV, as is determined by the nested loop cross-validation module 150, e.g., at the outer loop/inner loop module 156. Model performances with the best hyperparameters on 100 inner-testing sets are shown in FIGS. 8A-8C. Although these data were used to select hyperparameters and may suffer from overfitting, they represent results similar to those that would have been obtained if the regular non-nested CV approach was used for validation.

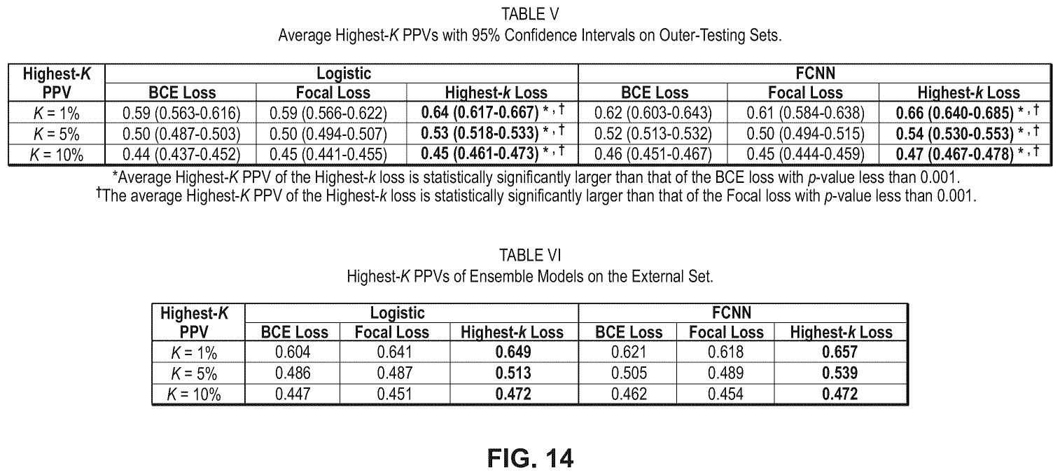

Table V (see, FIG. 14) better represents the true model performances as it is obtained from the outer-testing set when the best hyperparameters have been selected in the inner loops, determined by the outer loop/inner loop module 156. On average, the Highest-k loss improved the Highest 1% PPV by 0.05 (95% CI: 0.041-0.055), the Highest 5% PPV by 0.03 (95% CI: 0.024-0.032), and the Highest 10% PPV by 0.02 (95% CI: 0.016-0.021). For both model types, all improvements in the performance by the Highest-k loss over the BCE loss and the Focal loss were statistically significant.

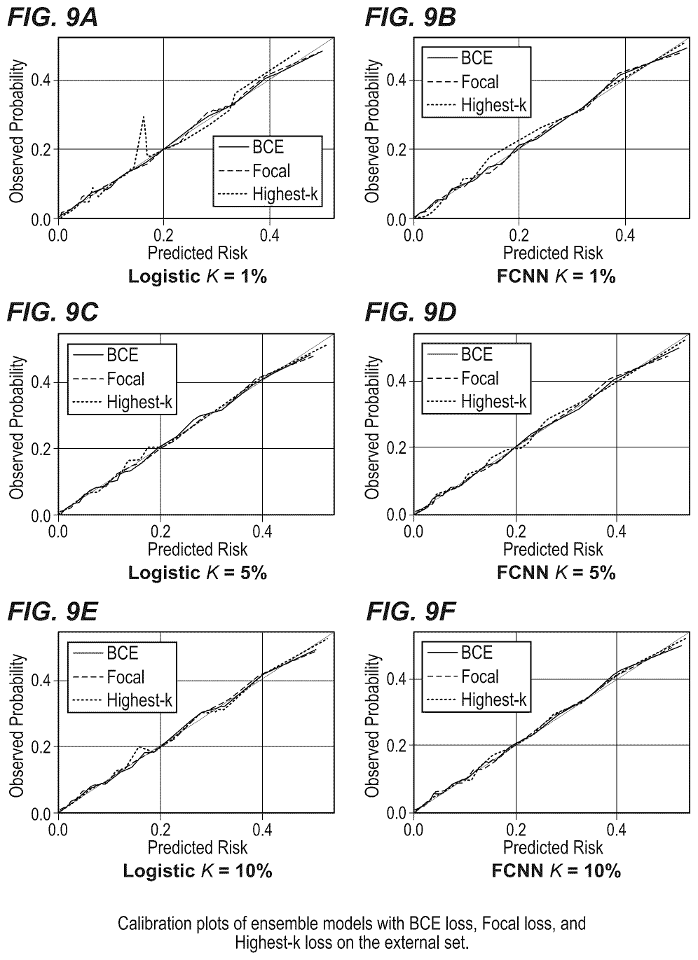

Finally, we obtained six ensemble models that targeted the highest K predictive risk scores for each K of 1%, 5%, and 10%. As shown in FIGS. 9A-9F, all ensemble models were well calibrated at high-risk levels, but models with the Highest-k loss were slightly less calibrated at the medium-risk and the low-risk levels. Table VI (see, FIG. 14) shows the performances of ensemble models at the target high-risk level. All models that used the Highest-k loss over-performed the BCE and Focal losses, with improvements ranging from 0.008 to 0.050.

The nested CV results on both the inner-and outer-testing sets show that the Highest-k loss can improve the precision by 0.02-0.05, corresponding to 4%-9% relative improvements, at all three selected high-risk levels. The external test results further demonstrate that the improvements in the precision among the highest predictive risk scores are generalizable.

### Comparisons in an Extreme Case

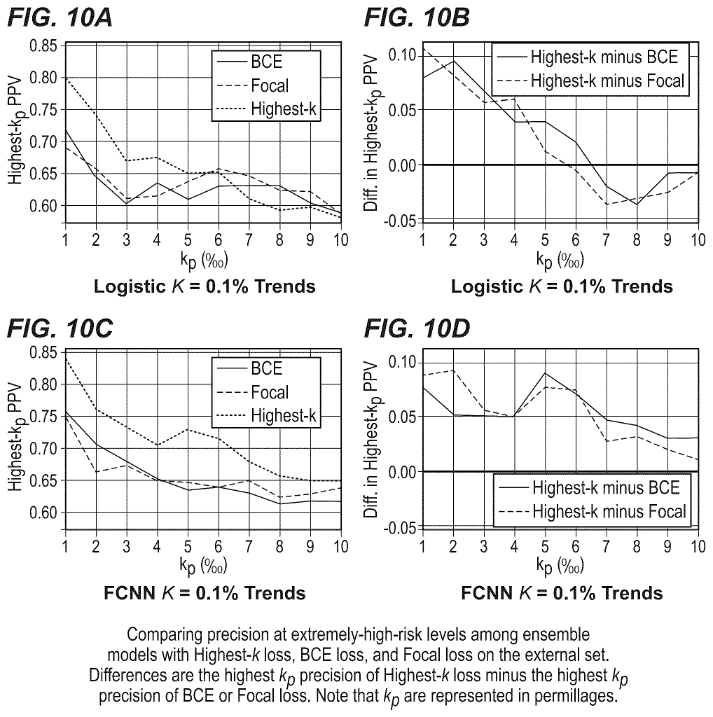

For the extreme situation where we focus on the extremely-high-risk level K=0.1%, we plot performances of the same model type with different loss functions together in FIGS. 10A-10D. For both model types, the Highest-k loss improved the precision by 0.05-0.10 around the highest 0.1% risk level. The proposed loss performed well even though its target parameter k overestimated the target proportion K. As expected, the performance advantages were narrowed as more samples were focused on and were reversed for the logistic model.

Thus, with these examples we show that a top-weighted loss function in the present techniques, in particular using a Highest-k loss, based on a differentiable estimation of the sorting operation. Different from the traditional binary prediction loss functions that try to minimize the number of false positives and false negatives at the same time for the entire target population, our proposed loss specifically targets precision by minimizing the number of false positives among observations with the highest scores.

The present disclosure provides systems and techniques that over many of the problems with traditional techniques. The toy example was an ideally designed dataset to allow us to preliminary verify the feasibility of the Highest-k loss techniques here and to analyze results on complicated real-world datasets, as we described. As are data shows, while traditional methods like BCE loss had a stable performance at a range of risk levels, our systems and methods described herein forced the prediction model to prioritize higher performance among observations with the highest scores. The model with the Highest-k loss assigned higher weights to x₂ to utilize the non-overlapping part of the distribution as shown in FIGS. 3A-3D, at the expense that more negative samples were misclassified when they were focused on. In contrast, x₂ was less useful in the model with the BCE loss compared to x₁ according to Table III (see, FIG. 12). When focusing on the highest-risk observations, the model with the BCE loss included more false positive samples and the precision decreased significantly.

In addition to the classical BCE loss, we also applied the Focal loss as the baseline loss function to address the class imbalance issue. But this method did not perform well in our experiments on the diabetes prediction task. Like other traditional class-balancing strategies, it assigns higher weights to minority samples, i.e., positive samples in our case, and lower weights to majority samples, i.e., negative samples. Such strategies essentially strengthen the effect of false negatives and weaken the effect of false positives. However, the false positives are what we care about since they reduce the precision among the highest kₚ scores and lead to over-treatment and resource-wasting in clinical practice.

The present techniques, while described in various examples, having vast practical applicability of the highest-K loss processes. Various different populations of data may be analyzed to identify a highest-K subpopulation and produce more accurate outcome predictions for that subpopulation over conventional systems. The result is improvement in accuracy of prediction computing systems, in particular those that rely upon trained machine learning prediction models. The further result is improvement in a healthcare providers allocation of resources to a subpopulation, for example, to ensure that a healthcare provider has sufficient resources for treating patients in the highest risk category.

As our experimental data shows, the Highest-k loss worked well at any target high-risk level when the target parameter k matched the target proportion K, while the precision at a higher-risk level benefited more from the Highest-k loss. Hence, in most clinical scenarios, we can choose k based on the proportion of the patients in the target population that can be followed up with, based on available resources.

The temperature parameter τ is another parameter that is to be specified. Generally, a smaller k should be paired with a larger τ to leave a fault-tolerant space for outliers. We gradually decreased τ during the training process such that the highest-k loss optimization module 152 put more attention on the highest k scores. The consistent improvements in the results over inner loop testing sets, outer loop testing sets, and the external set indicate that the Highest-k loss can be practically applied to improve the precision for the highest k predictive scores.

As discussed above, we simulated an extreme situation example, where only K=0.1% of the total population can be followed up with due to limited resources, and which might be the case of some severe disease or in areas with extremely scarce medical resources. In such scenarios, it may not be possible or it may be impractical to choose a k that matches K for many commonly used training batch sizes, such as 32, 64, or 128. Due to concerns about poor generalizations, we therefore provided example configurations of the high-risk patient module deploying highest-k loss optimization 116 in which output models were aggregated with the Highest-k loss with an overestimated target parameter k instead of having an extremely large training batch to match the target proportion. The evaluation results showed that performances at a specific risk level can also benefit from the Highest-k loss that targets other levels. For example, compared with BCE loss and Focal loss, the Highest-k loss performed better in a narrow range of extremely-high-risk levels. Thus, the methods enables may be used under extreme circumstances where only very few patients can be followed up with. When we are focusing on very few patients with an extremely small proportion of the total population, model selection and aggregation approaches such as nested CV and pruning can improve the robustness and the generalization of the Highest-k loss.

### ADDITIONAL EXEMPLARY ASPECTS

Aspect 1. A computer-implemented method for predicting disease risk in a sub-population, the method comprising: receiving characteristics data for each subject of a population, the characteristic data comprising demographic data and measured health data for each subject; providing the characteristics data to train a machine learning model, the machine learning model being trained to predict one or more biological outcomes for each subject of the population; during training of the machine learning model, imposing a resource limitation based loss function (“highest-k loss function”) that uses a soft sorting method to optimize accuracy of the machine learning model for a sub-population of the subjects who are at the highest risk for one or more biological outcomes in the population; during utilization of the machine learning model, providing characteristics data on a subsequent population of subjects and subsequent resource limitation data to the machine learning model, and the machine learning model determining one or more biological outcomes for a high-risk sub-population of the subsequent population and ranking the high-risk subpopulation for allocation of resources responsive to the one or more biological outcomes for the high-risk sub-population of the subsequent population; and storing the ranking.

Aspect 2. The computer-implemented method of Aspect 1, wherein the characteristics data comprises binary data for each subject.

Aspect 3. The computer-implemented method of Aspect 1, wherein the characteristics data comprises continuous data for each subject.

Aspect 4. The computer-implemented method of Aspect 1, wherein the resource limitation based loss function contains one or more available personnel data, facilities data such as data on available space for admitting a patient and for administering treatment to a patient and/or location data for determining which patients are in a vicinity of service, monitoring data such as available health monitoring equipment and equipment type, medical imager data such as medical imager type, and treatment data such as data on type and availability of any of a variety of types of treatments, from drug type and availability, and/or IV availability.

Aspect 5. The computer-implemented method of Aspect 1, wherein the machine learning model is a linear regression model.

Aspect 6. The computer-implemented method of Aspect 1, wherein the machine learning model is fully connected neural network.

Aspect 7. The computer-implemented method of Aspect 1, wherein imposing the resource limitation based loss function during the training of the model comprises; performing soft sorting on each of the subjects based on the risk or probability of one or more predicted biological outcomes; iteratively updating a soft sorting parameter to approach a sorted list of patients based on the risk or probability of one or more predicted biological outcomes; and integrating weights generated from the soft sorting into a loss function.

Aspect 8. The computer-implemented method of Aspect 7, wherein the soft sorting algorithm is NeuralSoft.

Aspect 9. The computer-implemented method of Aspect 7, wherein the soft sorting algorithm generates a soft sorting matrix, \hat{P}(s, τ), where s is score and τ>0 is a temperature parameter, where the weights generated from the soft sorting algorithm are expressed as:

$${\hat{w} ( s , k , τ )}_{j} = {\sum }_{i = 1}^{k} {\hat{P} ( s , τ )}_{ij} . \qquad ( 5 )$$

Aspect 10. The computer-implemented method of Aspect 7, further comprising: determining a weighted average as the highest-k loss function using,

$${ℒ}_{k , τ} ( s , y ) = \frac{{\sum }_{j = 1}^{N} {\hat{w} ( s , k , τ )}_{j} ( 1 - {y}_{j} )}{{\sum }_{j = 1}^{N} {\hat{w} ( s , k , τ )}_{j}} , \qquad ( 6 )$$

where k∈{1, 2, . . . , N} is called the target parameter, τ is the temperature parameter, N is the batch size, s is the predicted risk scores vector outputted by the risk prediction model, and y∈{0, 1}^{N} is the ground-truth labels vector and ŵ(s, k, τ) are weights.

Aspect 11. The computer-implemented method of Aspect 7, wherein the highest-k loss function is a binary cross-entropy (BCE) loss function expressed as,

$${ℒ}_{BCE , k , τ} ( s , y ) = \frac{{\sum }_{j = 1}^{N} {\hat{w} ( s , k , τ )}_{j} [ {y}_{j} log ( {s}_{j} ) + ( 1 - {y}_{j} ) log ( 1 - {s}_{j} ) ]}{{\sum }_{j = 1}^{N} {\hat{w} ( s , k , τ )}_{j}}$$

where k∈{1, 2, . . . , N} is called the target parameter, τ is the temperature parameter, N is the batch size, s∈[0, 1]^{N} is the predicted risk scores vector outputted by the risk prediction model, and y∈{0, 1}^{N} is the ground-truth labels vector and ŵ(s, k, τ) are weights.

Aspect 12. The computer-implemented method of Aspect 1, wherein the characteristic data comprises data comprises demographic data, monitored health data, medical assessment data, questionnaire data, diagnosis history data, treatment data, biomarker data, and/or medical images.

Aspect 13. A system for predicting disease risk in a sub-population, the system comprising: one or more processors; and one or more memories having stored thereon computer-executable instructions that, when executed by the one or more processors, cause the system to: receive characteristics data for each subject of a population, the characteristic data comprising demographic data and measured health data for each subject; provide the characteristics data to train a machine learning model, the machine learning model being trained to predict one or more biological outcomes for each subject of the population; during training of the machine learning model, impose a resource limitation based loss function (“highest-k loss function”) that uses a soft sorting process to optimize accuracy of the machine learning model for a sub-population of the subjects who are at the highest risk for one or more biological outcomes in the population; during utilization of the machine learning model, provide characteristics data on a subsequent population of subjects and subsequent resource limitation data to the machine learning model, and the machine learning model determines one or more biological outcomes for a high-risk sub-population of the subsequent population and ranking the high-risk subpopulation for allocation of resources responsive to the one or more biological outcomes for the high-risk sub-population of the subsequent population; and store the ranking.

Aspect 14. The system of Aspect 13, wherein the characteristics data comprises binary data for each subject.

Aspect 15. The system of Aspect 13, wherein the characteristics data comprises continuous data for each subject.

Aspect 16. The system of Aspect 13, wherein the resource limitation based loss function contains one or more available personnel data, facilities data such as data on available space for admitting a patient and for administering treatment to a patient and/or location data for determining which patients are in a vicinity of service, monitoring data such as available health monitoring equipment and equipment type, medical imager data such as medical imager type, and treatment data such as data on type and availability of any of a variety of types of treatments, from drug type and availability, and/or IV availability.

Aspect 17. The system of Aspect 13, wherein the machine learning model is a linear regression model.

Aspect 18. The system of Aspect 13, wherein the machine learning model is fully connected neural network.

Aspect 19. The system of Aspect 13, wherein the instructions to impose the resource limitation based loss function during the training of the model comprise instructions that, when executed by the one or more processors, cause the system to; perform soft sorting on each of the subjects based on the risk or probability of one or more predicted biological outcomes; iteratively update a soft sorting parameter to approach a sorted list of patients based on the risk or probability of one or more predicted biological outcomes; and integrate weights generated from the soft sorting into a loss function.

Aspect 20. The system of Aspect 13, wherein the characteristic data comprises data comprises demographic data, monitored health data, medical assessment data, questionnaire data, diagnosis history data, treatment data, biomarker data, and/or medical images.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the target matter herein.

Additionally, certain embodiments are described herein as including logic or a number of routines, subroutines, applications, or instructions. These may constitute either software (e.g., code embodied on a non-transitory, machine-readable medium) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations.

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

Those skilled in the art will recognize that a wide variety of modifications, alterations, and combinations can be made with respect to the above described embodiments without departing from the scope of the invention, and that such modifications, alterations, and combinations are to be viewed as being within the ambit of the inventive concept.

While the present invention has been described with reference to specific examples, which are intended to be illustrative only and not to be limiting of the invention, it will be apparent to those of ordinary skill in the art that changes, additions and/or deletions may be made to the disclosed embodiments without departing from the spirit and scope of the invention.

The foregoing description is given for clearness of understanding; and no unnecessary limitations should be understood therefrom, as modifications within the scope of the invention may be apparent to those having ordinary skill in the art.

What is claimed:

1. A computer-implemented method for predicting disease risk in a sub-population, the method comprising:
receiving characteristics data for each subject of a population, the characteristic data comprising demographic data and measured health data for each subject;
providing the characteristics data to train a machine learning model, the machine learning model being trained to predict one or more biological outcomes for each subject of the population;
during training of the machine learning model, imposing a resource limitation based loss function (“highest-k loss function”) that uses a soft sorting method to optimize accuracy of the machine learning model for a sub-population of the subjects who are at the highest risk for one or more biological outcomes in the population;
during utilization of the machine learning model, providing characteristics data on a subsequent population of subjects and subsequent resource limitation data to the machine learning model, and the machine learning model determining one or more biological outcomes for a high-risk sub-population of the subsequent population and ranking the high-risk subpopulation for allocation of resources responsive to the one or more biological outcomes for the high-risk sub-population of the subsequent population; and
storing the ranking.

2. The computer-implemented method of claim 1, wherein the characteristics data comprises binary data for each subject.

3. The computer-implemented method of claim 1, wherein the characteristics data comprises continuous data for each subject.

4. The computer-implemented method of claim 1, wherein the resource limitation based loss function contains one or more available personnel data, facilities data such as data on available space for admitting a patient and for administering treatment to a patient and/or location data for determining which patients are in a vicinity of service, monitoring data such as available health monitoring equipment and equipment type, medical imager data such as medical imager type, and treatment data such as data on type and availability of any of a variety of types of treatments, from drug type and availability, and/or IV availability.

5. The computer-implemented method of claim 1, wherein the machine learning model is a linear regression model.

6. The computer-implemented method of claim 1, wherein the machine learning model is fully connected neural network.

7. The computer-implemented method of claim 1, wherein imposing the resource limitation based loss function during the training of the model comprises;
performing soft sorting on each of the subjects based on the risk or probability of one or more predicted biological outcomes;
iteratively updating a soft sorting parameter to approach a sorted list of patients based on the risk or probability of one or more predicted biological outcomes; and
integrating weights generated from the soft sorting into a loss function.

8. The computer-implemented method of claim 7, wherein the soft sorting algorithm is NeuralSoft.

9. The computer-implemented method of claim 7, wherein the soft sorting algorithm generates a soft sorting matrix, \hat{P}(s, τ), where s is score and τ>0 is a temperature parameter, where the weights generated from the soft sorting algorithm are expressed as:
$${\hat{w} ( s , k , τ )}_{j} = {\sum }_{i = 1}^{k} {\hat{P} ( s , τ )}_{ij} . \qquad ( 5 ) .$$

10. The computer-implemented method of claim 7, further comprising:
determining a weighted average as the highest-k loss function using,
$${ℒ}_{k , τ} ( s , y ) = \frac{{\sum }_{j = 1}^{N} {\hat{w} ( s , k , τ )}_{j} ( 1 - {y}_{j} )}{{\sum }_{j = 1}^{N} {\hat{w} ( s , k , τ )}_{j}} , \qquad ( 6 )$$
where k∈{1, 2, . . . , N} is called the target parameter, τ is the temperature parameter, N is the batch size, s is the predicted risk scores vector outputted by the risk prediction model, and y∈{0, 1}^{N} is the ground-truth labels vector and ŵ(s, k, τ) are weights.

11. The computer-implemented method of claim 7, wherein the highest-k loss function is a binary cross-entropy (BCE) loss function expressed as,
$${ℒ}_{BCE , k , τ} ( s , y ) = \frac{{\sum }_{j = 1}^{N} {\hat{w} ( s , k , τ )}_{j} [ {y}_{j} log ( {s}_{j} ) + ( 1 - {y}_{j} ) log ( 1 - {s}_{j} ) ]}{{\sum }_{j = 1}^{N} {\hat{w} ( s , k , τ )}_{j}}$$
where k∈{1, 2, . . . , N} is called the target parameter, τ is the temperature parameter, N is the batch size, s∈[0, 1]^{N} is the predicted risk scores vector outputted by the risk prediction model, and y∈{0, 1}^{N} is the ground-truth labels vector and ŵ(s, k, τ) are weights.

12. The computer-implemented method of claim 1, wherein the characteristic data comprises data comprises demographic data, monitored health data, medical assessment data, questionnaire data, diagnosis history data, treatment data, biomarker data, and/or medical images.

13. A system for predicting disease risk in a sub-population, the system comprising:
one or more processors; and
one or more memories having stored thereon computer-executable instructions that, when executed by the one or more processors, cause the system to:
receive characteristics data for each subject of a population, the characteristic data comprising demographic data and measured health data for each subject;
provide the characteristics data to train a machine learning model, the machine learning model being trained to predict one or more biological outcomes for each subject of the population;
during training of the machine learning model, impose a resource limitation based loss function (“highest-k loss function”) that uses a soft sorting process to optimize accuracy of the machine learning model for a sub-population of the subjects who are at the highest risk for one or more biological outcomes in the population;
during utilization of the machine learning model, provide characteristics data on a subsequent population of subjects and subsequent resource limitation data to the machine learning model, and the machine learning model determines one or more biological outcomes for a high-risk sub-population of the subsequent population and ranking the high-risk subpopulation for allocation of resources responsive to the one or more biological outcomes for the high-risk sub-population of the subsequent population; and
store the ranking.

14. The system of claim 13, wherein the characteristics data comprises binary data for each subject.

15. The system of claim 13, wherein the characteristics data comprises continuous data for each subject.

16. The system of claim 13, wherein the resource limitation based loss function contains one or more available personnel data, facilities data such as data on available space for admitting a patient and for administering treatment to a patient and/or location data for determining which patients are in a vicinity of service, monitoring data such as available health monitoring equipment and equipment type, medical imager data such as medical imager type, and treatment data such as data on type and availability of any of a variety of types of treatments, from drug type and availability, and/or IV availability.

17. The system of claim 13, wherein the machine learning model is a linear regression model.

18. The system of claim 13, wherein the machine learning model is fully connected neural network.

19. The system of claim 13, wherein the instructions to impose the resource limitation based loss function during the training of the model comprise instructions that, when executed by the one or more processors, cause the system to;
perform soft sorting on each of the subjects based on the risk or probability of one or more predicted biological outcomes;
iteratively update a soft sorting parameter to approach a sorted list of patients based on the risk or probability of one or more predicted biological outcomes; and
integrate weights generated from the soft sorting into a loss function.

20. The system of claim 13, wherein the characteristic data comprises data comprises demographic data, monitored health data, medical assessment data, questionnaire data, diagnosis history data, treatment data, biomarker data, and/or medical images.
